(19) United States
(12) Patent Application Publication
(10) Pub. No.: US 2023/0165884 A1
(43) Pub. Date: Jun. 1, 2023

(54) METHOD AND COMPOSITION FOR TREATING ALZHEIMERS

(71) Applicant: Cognitive Synergy Corporation, San Diego, CA (US)

(72) Inventor: Asha Amritraj, San Diego, CA (US)

(73) Assignee: Cognitive Synergy Corporation, San Diego, CA (US)

(21) Appl. No.: 18/059,383

(22) Filed: Nov. 28, 2022

Related U.S. Application Data
(60) Provisional application No. 63/283,800, filed on Nov. 29, 2021.

(51) Int. Cl. A61K 31/7048; A61K 31/366; A61K 45/06

(52) U.S. Cl. CPC A61K 31/7048; A61K 31/366; A61K 45/06

(57) ABSTRACT

Compositions and methods for treating Alzheimer's disease are provided. An exemplary embodiment is a pharmaceutical composition for treatment of Alzheimer's disease in a patient. The pharmaceutical composition includes an effective dose of at least one of orientin, vitexin and bergenin.

### CROSS REFERENCE TO PRIOR APPLICATION

This application claims the benefit of U.S. Provisional Patent Application No. 63/283,800, entitled as “METHOD AND COMPOSITION FOR TREATING ALZHEIMERS”, filed Nov. 29, 2021, which is incorporated by reference in its entirety.

### FIELD OF THE INVENTION

This disclosure relates to methods and treatments for cognitive disorders such as Alzheimer's disease.

### BACKGROUND

Alzheimer's disease is a common form of dementia. Although it may begin earlier in some cases, symptoms of Alzheimer's disease generally begin in individuals 65 years and older. The disease is characterized by a slow and gradual cognitive decline that manifests early as simple memory loss. Later, the individual gradually loses cognitive function to the point that they are entirely dependent on caregivers. In late-stage Alzheimer's, the individual often loses their ability to speak.

Despite widespread and omnipresent suffering from Alzheimer's disease, there is no cure. Indeed, there are only a handful of therapies to treat Alzheimer's disease, and they only offer marginal relief. And this is all while the cost of Alzheimer's disease in money, time, and anguish takes an increasing toll on the world population as life expectancy increases.

Although the cause of Alzheimer's disease is not completely understood, there is increasing evidence that Aβ protein (abeta) and tau protein play a significant role. For instance, Alzheimer's disease is characterized by the deposition of Aβ amyloid fibrils, which are extracellular deposits of Aβ protein plaques in the gray matter of the brain. Alzheimer's disease is further characterized by the deposition of neurofibrillary tangles, which are abnormal accumulations inside neurons of tau protein.

### SUMMARY

Methods and compositions for the treatment of Alzheimer's disease are provided. An exemplary embodiment is a composition. The composition includes an effective dose of at least one of orientin, vitexin and bergenin. The effective dose may include orientin and vitexin. The effective dose may comprise orientin and bergenin. The effective dose of aconitine and bergenin may include an orientin concentration of between about 10 micromoles per liter to 60 micromoles per liter. The effective dose of aconitine and bergenin may include a bergenin volume of between about 25 micromoles per liter to 100 micromoles per liter. The effective dose may comprise vitexin and bergenin where the effective dose further includes a vitexin concentration of between about 10 micromoles per liter to 60 micromoles per liter. The effective dose may include orientin, vitexin, and bergenin. The effective dose of orientin, vitexin, and bergenin may further include an orientin concentration of between about 10 micromoles per liter to 60 micromoles per liter. The effective dose of aconitine, orientin, and bergenin may further comprise a bergenin concentration of between about 25 micromoles per liter to 100 micromoles per liter and a vitexin concentration of between about 10 micromoles per liter to 60 micromoles per liter.

A general aspect is a method for the treatment of a patient. The method includes administering an effective dose of at least one of orientin, vitexin and bergenin. The effective dose may include orientin and vitexin. The effective dose may include orientin and bergenin. The effective dose of orientin and bergenin may include an orientin concentration of between about 10 micromoles per liter to 60 micromoles per liter. The effective dose of orientin and bergenin may include a concentration of between about 25 micromoles per liter to 100 micromoles per liter. The effective dose may include vitexin and bergenin. The effective dose may include orientin, vitexin, and bergenin. The effective dose may further include an orientin concentration of between about 10 micromoles per liter to 60 micromoles per liter. The effective dose may further include a bergenin concentration of between about 25 micromoles per liter to 100 micromoles per liter. The effective dose may further include a vitexin concentration of between about 10 micromoles per liter to 60 micromoles per liter.

Another general aspect is a composition for treatment of a patient. The composition includes an effective dose comprising orientin, vitexin and bergenin where the effective dose further includes an orientin concentration of between about 10 micromoles per liter to 60 micromoles per liter. The effective dose may further include a bergenin concentration of between about 25 micromoles per liter to 100 micromoles per liter and a vitexin concentration of between about 10 micromoles per liter to 60 micromoles per liter.

### BRIEF DESCRIPTION OF THE DRAWINGS

FIG. 1 is electron microscopy images of aggregated amyloid beta 1-42 (Aβ₁₋₄₂) under Synaptecx-227 treatment for various times. Image A is an electron microscopy image of Aβ₁₋₄₂ after 48 hours incubation at 37° C. Image B is an electron microscopy image of Aβ₁₋₄₂ after 48 hours incubation at 37° C. with Synaptecx-227. Image C is an electron microscopy image of Aβ₁₋₄₂ after 5 days incubation at 37° C. Image D is an electron microscopy image of Aβ₁₋₄₂ after 5 days incubation at 37° C. with Synaptecx-227.

FIG. 2 is a graph of thioflavin T (ThT) florescence at 490 nm for a sample of Aβ₁₋₄₂ after various periods of incubation at 37° C. without Synaptecx-227, with Synaptecx-227, and a control of phosphate-buffered saline.

FIG. 3 is a graph of thioflavin T (ThT) florescence at 490 nm for pre-aggregated Aβ₁₋₄₂ samples for 24 hours and then various periods of incubation at 37° C. without Synatptecx-227, with Synaptecx-227, and a control of phosphate-buffered saline.

FIG. 4 is live/dead assay showing viability of neurons. The image on the left is a control. The middle image are neurons treated with Aβ₂₅₋₃₅. The image on the right shows neurons that were treated with Synaptecx-227 and Aβ₂₅₋₃₅ for 24 hours.

FIG. 5 is a bar graph showing a percentage of mouse hippocampal neurons after a 48 hr period of treatment. The controls are shown in the darker bars. The control sample 505 on the far left has neurons that were treated with Aβ₂₅₋₃₅ at a concentration of 20 μM The lighter bars indicate subsequent treatment with Synaptecx-227. The fourth sample from the left contains neurons that were treated with Aβ₁₋₄₂ at a concentration of 10 μM. All measurements were taken after 48 hours.

FIG. 6 is a bar graph showing the effect of Synaptecx-227 on a percentage of tau protein fibrillization.

FIG. 7 is an image of a western blot indicating the disruption of Aβ fibrils. The two control samples on the left are treated with Aβ₁₋₄₂ at 25 μM. The two samples on the right are additionally treated with Synatptecx-227.

FIG. 8 shows an effect of various fractions of Sample A, where Synaptecx-227 has been fractionated into Sample A and Sample B, on Aβ fibrils.

FIG. 9 shows an effect of various fractions of Sample B, where Synaptecx-227 has been fractionated into Sample A and Sample B, on Aβ fibrils.

FIG. 10 is a LCMS mass spectrometry (MS)—analysis for the A14 fraction of Sample A. An LCMS graph is shown on top. On the bottom is an MS of a fraction taken at 3.4 minutes on the LCMS.

FIG. 11 is a probable structure of the fraction taken at 3.4 minutes on the LCMS for Sample A.

FIG. 12 is an LCMS-mass spectrometry (MS) spectra for the A14 fraction of Sample A. On the bottom is an MS of a fraction taken at 1.5 minutes on the LCMS.

FIG. 13 is a probable structure of the fraction taken at 1.5 minutes on the LCMS for Sample B.

FIG. 14 is an electron microscopy image of Aβ₁₋₄₂ protein aggregation when incubated for 24 hrs at 37° C.

FIG. 15 is an electron microscopy image of Aβ₁₋₄₂ protein that is treated with the A2 fraction from Sample A.

FIG. 16 is an electron microscopy image of Aβ₁₋₄₂ protein that is treated with the A14 fraction from Sample A.

FIG. 17 is an electron microscopy image of Aβ₁₋₄₂ protein that is treated with the B2 fraction from Sample B.

FIG. 18 is an analysis showing a percentage of Aβ₁₋₄₂ protein fibrillization using thioflavin T (ThT) florescence in various samples. The sample on the left is a control of Aβ₁₋₄₂ protein. The second sample from the left is Aβ₁₋₄₂ protein treated with Synaptecx-227. The third sample from the left is Aβ protein treated with 10 mM Aconitine. And the fourth sample from the left is Aβ₁₋₄₂ protein treated with 20 mM Aconitine.

FIG. 19 is an analysis showing an amount of Aβ₁₋₄₂ protein fibrillization using thioflavin T (ThT) fluorescence in various samples. The sample on the left is a control of Aβ₁₋₄₂ protein. The second sample from the left is Aβ₁₋₄₂ protein treated with 200 mM of bergenin. The third sample from the left is Aβ₁₋₄₂ protein treated with 300 mM bergenin.

FIG. 20A is an LCMS-mass spectrometry (MS) spectra for a Synaptecx-227 sample (Sample A) showing retention time (rt) in minutes on the x-axis and mass to charge ratio (mz) on the y-axis.

FIG. 20B is a chromatogram of the LCMS shown in FIG. 20A.

FIG. 21A is an LCMS-mass spectrometry (MS) spectra for fraction 2 of the Synaptecx-227 sample (Sample A2) showing retention time (rt) in minutes on the x-axis and mass to charge ratio (mz) on the y-axis.

FIG. 21B is a chromatogram of the LCMS shown in FIG. 21A.

FIG. 22A is an LCMS-mass spectrometry (MS) spectra for a first commercially available Ekanveer Ras sample showing retention time (rt) in minutes on the x-axis and mass to charge ratio (mz) on the y-axis.

FIG. 22B is a chromatogram of the LCMS shown in FIG. 22A.

FIG. 23A is an LCMS-mass spectrometry (MS) spectra for a second commercially available Ekanveer Ras sample showing retention time (rt) in minutes on the x-axis and mass to charge ratio (mz) on the y-axis.

FIG. 23B is a chromatogram of the LCMS shown in FIG. 23A.

FIG. 24A is an LCMS-mass spectrometry (MS) spectra for an orientin sample showing retention time (rt) in minutes on the x-axis and mass to charge ratio (mz) on the y-axis.

FIG. 24B is a chromatogram of the LCMS shown in FIG. 24A.

FIG. 25A is an LCMS-mass spectrometry (MS) spectra for a vitexin-4-′rhamniside sample showing retention time (rt) in minutes on the x-axis and mass to charge ratio (mz) on the y-axis.

FIG. 25B is a chromatogram of the LCMS shown in FIG. 25A.

FIG. 26 shows two mass spectra for comparison. The first mass spectra of the identified orientin portion of Sample A (Synaptecx-227) is displayed from 0 to 1 on the x axis. The second mass spectra of commercial orientin is displayed from 0 to −1 on the x-axis.

FIG. 27 shows two mass spectra for comparison. The first mass spectra of the identified vitexin portion of Sample A (Synaptecx-227) is displayed from 0 to 1 on the x axis. The second mass spectra of commercial vitexin-4-′rhamnoside is displayed from 0 to −1 on the x-axis.

FIG. 28 shows the result of a fibrillization assay of various compositions.

FIG. 29 shows the result of a cell survival assay of various compositions.

### DETAILED DESCRIPTION

The disclosed subject matter is a composition for treating Alzheimer's disease. A treatment composition is made from components that are derived from Sameer Panag Ras, Mahavat Vidhwansan Ras, Sutshekhar Ras and Ekangveer Ras. A composition labeled Synaptecx-227 is prepared from the treatment composition for the treatment of Alzheimer's disease or any other disease such as various neurologic diseases. Synaptecx-227 has been shown to reduce Aβ amyloid fibrils and tau protein. However, Synaptecx-227 contains a vast number of compounds. It is not previously known as to which of the compounds, which make up Synaptecx-227, contribute to defibrillation of Aβ and tau protein. The disclosed subject matter is an Alzheimer's treatment, that is based on identification of compounds that make up Synaptecx-227 and reduces Aβ amyloid fibrils and accumulations of tau protein.

In an exemplary embodiment, the composition comprises various combinations of orientin, vitexin and bergenin, which are identified as bioactive metabolites of Synaptecx-227. Synaptecx-227 is shown to reduce incidence of Aβ amyloid fibrils and neurofibrillary tangles that are created by Aβ protein and tau protein respectively. Orientin has a molecular weight of 448.38 g/mol and has the molecular formula C₂₁H₂₀O₁₁. Vitexin has a molecular weight of 432.38 g/mol and has the molecular formula C₂₁H₂₀O₁₀. Bergenin has a molecular weight of 328.27 g/mol and has the molecular formula C₁₄H₁₆O₉.

Orientin, vitexin, and bergenin were detected as possible targets based on identification via Liquid Chromatography—mass Spectrometry (LC-MS) from Synaptecx-227 and verified via mass spectrometric (MS) analysis. Aβ₁₋₄₂ defibrillation analysis and Aβ₁₋₄₂ cell survival were performed for various combinations of the above identified targets.

Aconitine, swatinine and bergenin were also identified as possible targets from Synaptecx-227 via Liquid Chromatography—mass Spectrometry (LC-MS) and identified via mass spectrometric (MS) analysis. The various compounds were incubated with Aβ₁₋₄₂ for 24 hours at 37° C. to determine the effect of compounds on defibrillation. Further, testing was done to determine an effect of the compounds on tau protein. As such, aconitine, swatinine and bergenin are identified as reducing a percentage of Aβ protein aggregation as compared to a control and/or reducing a percentage of tau protein accumulation as compared to a control.

In an exemplary embodiment, the composition comprises various combinations of aconitine, swatinine and bergenin, which are identified as reducing the incidence of Aβ amyloid fibrils and neurofibrillary tangles, which are created by Aβ protein and tau protein, respectively. Aconitine has a molecular weight of 645.74 g/mol and has the molecular formula C₃₄H₄₇NO₁₁ Swatinine has a molecular weight of 483.6 g/mol and has the molecular formula C₂₅H₄₁NO₈. Bergenin has a molecular weight of 328.27 g/mol and has the molecular formula C₁₄H₁₆O₉.

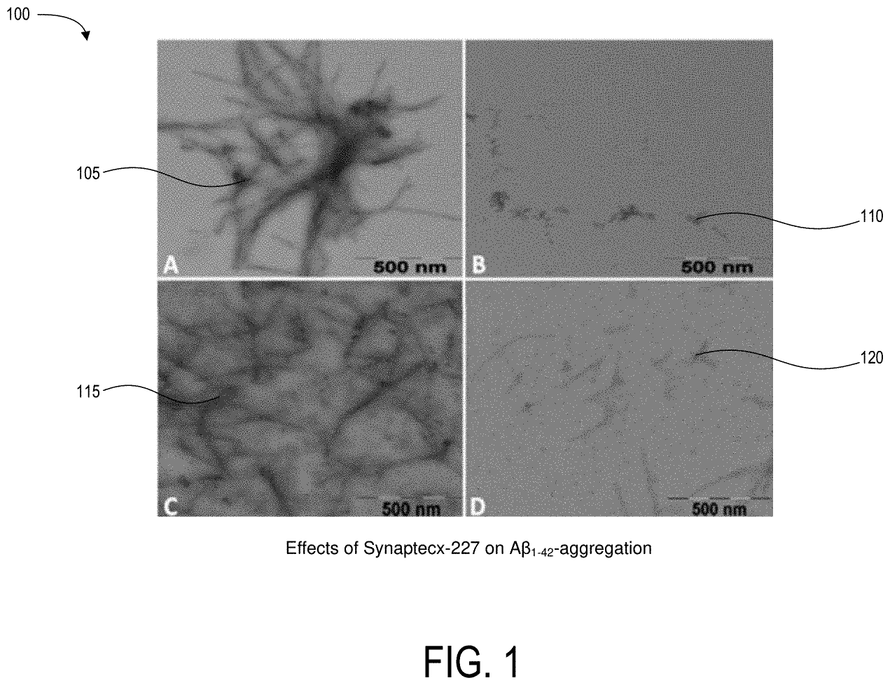

Referring to FIG. 1, FIG. 1 is electron microscopy images 100 of aggregated Aβ₁₋₄₂ under Synaptecx-227 treatment for various times. Image A is an electron microscopy image of Aβ₁₋₄₂ after 48 hours incubation at 37° C. Image B is an electron microscopy image of Aβ₁₋₄₂ after 48 hours incubation at 37° C. with Synaptecx-227. Image C is an electron microscopy image of Aβ₁₋₄₂ after 5 days incubation at 37° C. Image D is an electron microscopy image of Aβ₁₋₄₂ after 5 days incubation at 37° C. with Synaptecx-227.

Image B is conducted under similar conditions to image A, and with the addition of Synaptecx-227 treatment. Image A shows substantial fibril like structures 105. The fibril like structures 110 in image B, however, are noticeably smaller. Accordingly, image B shows substantially less aggregation than image A. Like image A, where the Aβ₁₋₄₂ incubated for 48 hours, image C, where Aβ₁₋₄₂ incubated for 5 days, shows substantial fibril-like structures 115. Likewise, image D shows substantially less aggregation when compared to image C with a marked decrease in the fibril-like structures 120. The results of the microscopic images shown in FIG. 1 show that the presence of Synaptecx-227 reduces the rate of Aβ₁₋₄₂ aggregation.

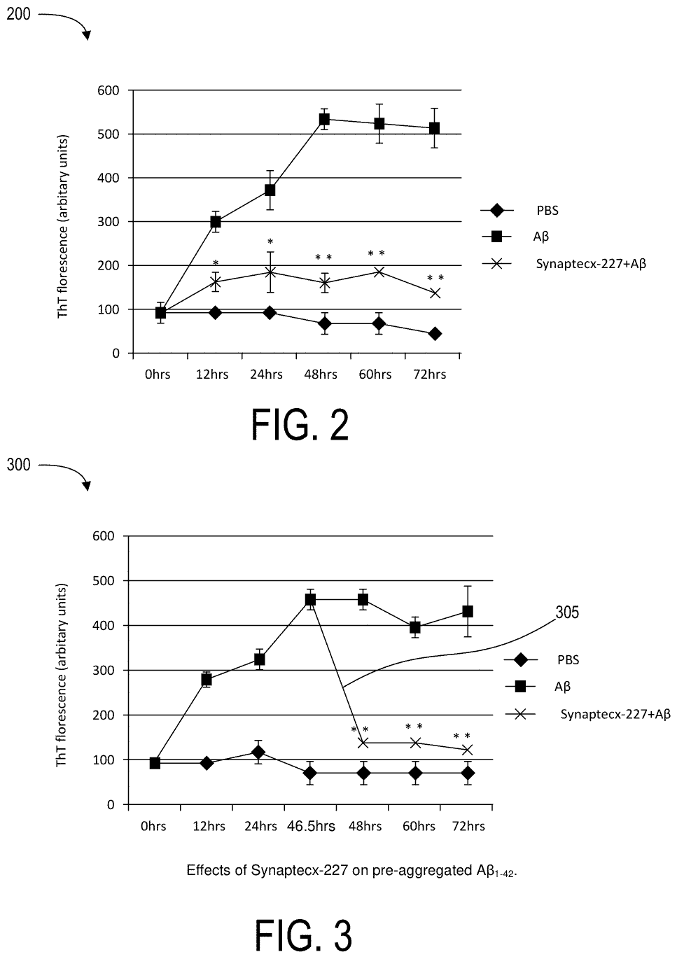

Referring to FIG. 2, FIG. 2 is a graph 200 of thioflavin T (ThT) fluorescence at 490 nm for a sample of Aβ₁₋₄₂ after various periods of incubation at 37° C. without Synaptecx-227, with Synaptecx-227, and a control of phosphate-buffered saline (PBS). The various times include 0 hours, 12 hours, 24 hours, 48 hours, 60 hours, and 72 hours. The amount of fluorescence indicates an amount of Aβ₁₋₄₂ aggregation in the sample. The PBS control may be treated as a baseline.

The graph of Aβ₁₋₄₂ by itself shows an increase in fluorescence, which indicates an increase in Aβ₁₋₄₂ aggregation from 0 to 48 hours of incubation. After 48 hours of incubation, the amount of Aβ₁₋₄₂ aggregation levels off. When compared to the sample with both Aβ₁₋₄₂ and Synaptecx-227, the sample with just Aβ₁₋₄₂ had a substantially increased amount of Aβ₁₋₄₂ aggregation. The sample of both Aβ₁₋₄₂ and Synaptecx-227 increases in Aβ₁₋₄₂ aggregation from 0 hours to 12 hours and then levels off.

Referring to FIG. 3, FIG. 3 is a graph 300 of thioflavin T (ThT) fluorescence at 490 nm for pre-aggregated Aβ₁₋₄₂ samples for 46.5 hours and then various periods of incubation at 37° C. without Synatptecx-227, with Synaptecx-227, and a PBS control. The pre-aggregation occurs from 0 hours to 46.5 hours on the graph, which is why the samples have substantially the same fluorescence values through 46.5 hours. At the 48-hour mark, after 1.5 hours of incubation with Synatptecx-227, the sample with both Aβ₁₋₄₂+Synatptecx-227 shows a substantial decrease in fluorescence as compared to the Aβ₁₋₄₂ sample.

In comparing FIG. 2 to FIG. 3, the result for Aβ₁₋₄₂ is largely the same. And even though the Aβ₁₋₄₂+Synaptecx-227 in FIG. 3 is pre-aggregated in just Aβ₁₋₄₂ for 46.5 hours, the results from 48 hours appear to be substantially the same between FIG. 2 and FIG. 3. Also of note is the 1.5 hour difference 305 between the 46.5 hour and 48 hour measurements for the Aβ₁₋₄₂+Synaptecx-227 in FIG. 3. It can be inferred from the rapid change from 46.5 hours to 48 hours that defibrillation of aggregated Aβ₁₋₄₂ occurs relatively quickly in the presence of Synaptecx-227.

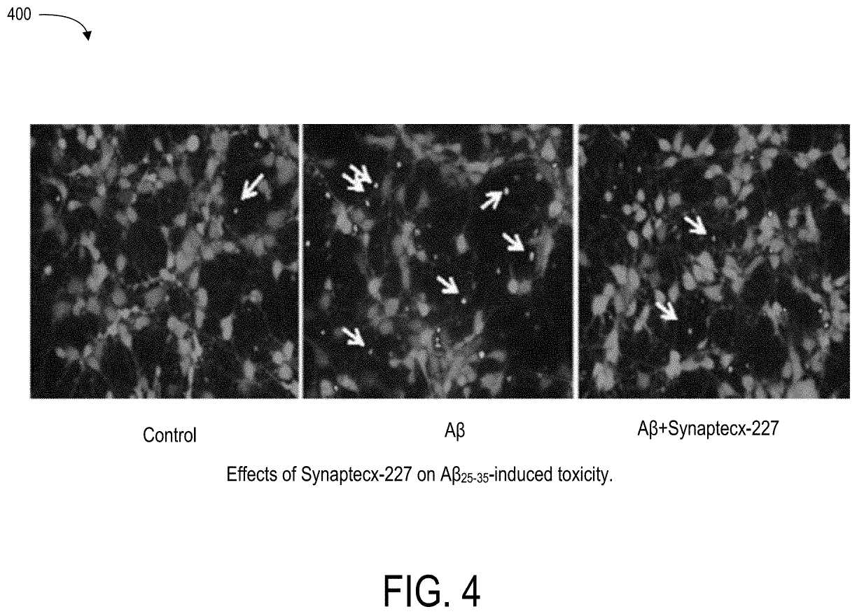

Referring to FIG. 4, FIG. 4 is a live/dead assay 400 showing viability of mouse hippocampal neurons. The image on the left is a control. The middle image are neurons treated with Aβ₂₅₋₃₅. The image on the right shows neurons that were treated with Aβ₂₅₋₃₅ and Synaptecx-227 for 24 hours. Live cells are brightly colored and dead cells (increased EthD-1 positive) are indicated with arrows pointing at dots. The control shows a large amount of bright area. Further, the control shows a small number of dead cells as indicated by the dots and arrow.

The sample with just Aβ₂₅₋₃₅ for 24 hours is in the middle image. It has noticeably less bright area, which indicates fewer living cells. Further, the image of just the Aβ₂₅₋₃₅ sample has far more dead cells than the control. When compared to the sample of both Aβ₂₅₋₃₅ and Synaptecx-227 (rightmost image), the sample of just Aβ₂₅₋₃₅ appears to have far more dead cells and fewer live cells.

The sample on the right that was treated with Aβ₂₅₋₃₅ and Synaptecx-227 for 24 hours. It shows a substantial number of live cells on par with the control. The number of dead cells in the image on the right is greater than the control, but less than the sample with just Aβ₂₅₋₃₅.

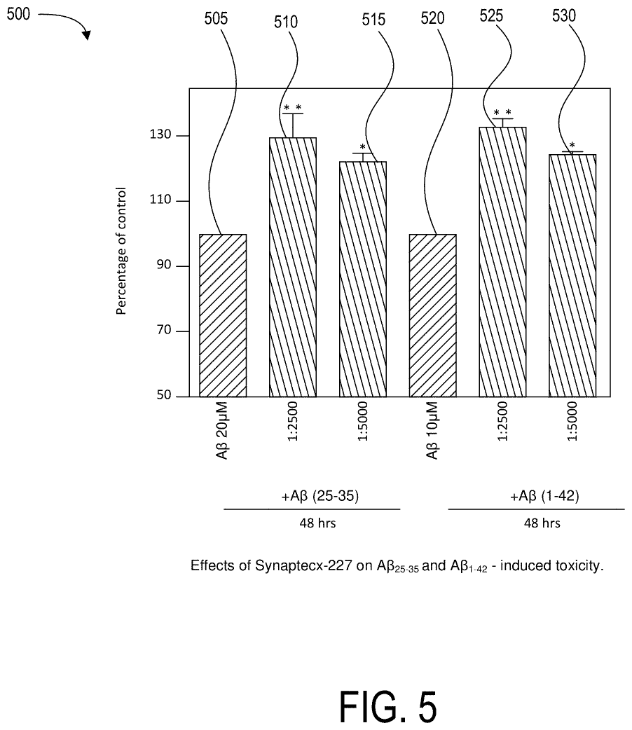

Referring to FIG. 5, FIG. 5 is a bar graph 500 showing a percentage of mouse hippocampal neurons after a period of treatment. Cell viability was assessed by MTT reduction. The controls (control sample 505 and control sample 520) are shown with the same shading. The control sample 505 on the far left has neurons that were treated with Aβ₂₅₋₃₅ at a concentration of 20 μM. The four other bars with similar shading indicate concurrent treatment with Synaptecx-227. The three samples indicated by the bars on the right contain neurons that were treated with Aβ₁₋₄₂ at a concentration of 10 μM. All measurements were taken after 48 hours.

Looking at the control sample 505 on the far left with Aβ₂₅₋₃₅ at a concentration of 20 μM, its cell viability is normalized to a value of 100. The next sample 510 to the right is additionally treated with a 1:2500 ratio of Synaptecx-227. The third sample 515 from the left is additionally treated with a 1:5000 ratio of Synaptecx-227. Both sample 510 and sample 515 have significantly increased viability as compared to the control sample 505. Sample 510 has a cell viability of approximately 129 and sample 515 has a cell viability of approximately 122 as compared to a cell viability of 100 for the control sample 505. Sample 515, which has half the concentration of Synaptecx-227 as sample 510, shows slightly less cell viability than sample 510. Accordingly, it may be inferred that increasing the concentration of Synaptecx-227 is directly correlated with an increase in cell viability.

The three samples on the right, which are all treated with Aβ₁₋₄₂ at a concentration of 10 μM, show similar results. The control sample 520, which is just Aβ₁₋₄₂, is set to a value of 100. Sample 525, which is also treated with a 1:2500 ratio of Synaptecx-227, has a cell viability of approximately 130. Sample 530, which is also treated with a 1:5000 ratio of Synaptecx-227, shows a cell viability of approximately 123. Accordingly, the effect of Synaptecx-227 is similar in samples treated with both Aβ₂₅₋₃₅ and A(31.42. Cell viability appears in all cases to increase by 20-30% when concurrently treated with Synaptecx-227. Cell viability increases significantly more for sample 525, which has a Synaptecx-227 concentration of 1:2500 as compared to sample 530, which has a Synaptecx-227 concentration of 1:5000.

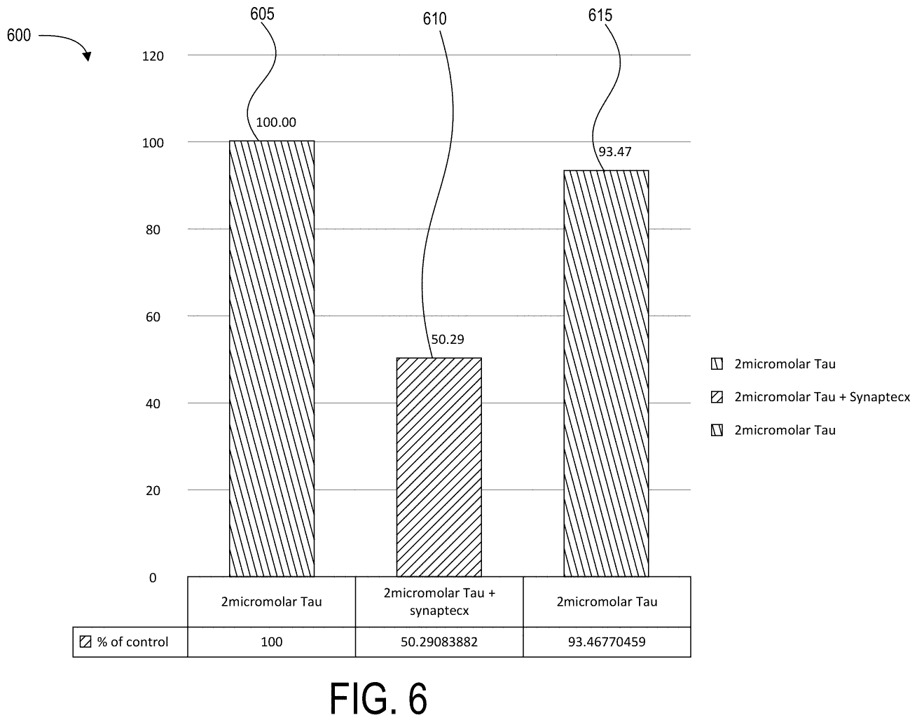

Referring to FIG. 6, FIG. 6 is a bar graph 600 showing the effect of Synaptecx-227 on a percentage of tau protein fibrillization. Each of the samples have a concentration of 2 mM tau protein. A control sample 605 is shown on the left side of the bar graph 600. The value of tau accumulation for the control sample 605 is normalized to 100.00. Sample 615 on the right side of the bar graph also has just tau protein. Its value is measured at 93.47.

Sample 610 shown in the middle of the bar graph 600 contains 2 mM tau protein and Synaptecx-227. The value for tau accumulation for sample 610 is 50.29. Accordingly, treatment with Synaptecx-227 substantially reduces accumulations of tau protein.

One or more compounds identified in Synaptecx-227 to be active in breaking up accumulated tau protein, preventing tau protein accumulation, or otherwise reducing tau protein accumulation in solution may be used alone to treat Alzheimer's disease. Further, one or more identified compounds from Synaptecx-227 may be combined with one another at various concentrations to meet or even surpass the effectiveness of Synaptecx-227. In various embodiments, the one or more identified compounds from Synaptecx-227 that are active in reducing accumulation of tau protein may be combined with one or more compounds identified from Synaptecx-227 that reduce accumulation of Aβ₂₅₋₃₅ and or Aβ₁₋₄₂.

In an exemplary embodiment, an effective dose of a compound identified to be active in Synaptecx-227 in reducing accumulation of tau protein may be administered to an individual to treat Alzheimer's disease. In various embodiments, more than one compound, which are identified to be active in Synaptecx-227 in reducing accumulation of tau protein, may be combined at an effective concentration and dose to be administered to an individual to treat Alzheimer's disease.

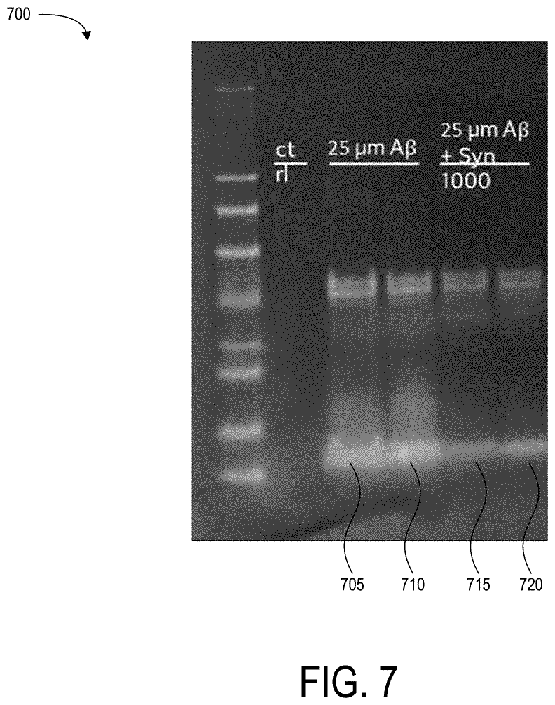

Referring to FIG. 7, FIG. 7 is an image 700 of a western blot indicating the disruption of Aβ fibrils The two control samples on the left, sample 705 and sample 710, are treated with Aβ₁₋₄₂ at 25 μM. The two samples on the right, sample 715 and sample 720, are additionally treated with Synatptecx-227. The two control samples show stronger intensities for Aβ than samples 715 and 720. Accordingly, treatment of samples containing Aβ with Synatptecx-227 may reduce expression of Aβ.

In a study, the three compounds aconitine, swatinine, and bergenin, were identified from Synatptecx-227 and determined to take part in reducing harmful accumulations of Aβ and/or tau protein. An embodiment of the disclosed subject matter is a process to administer an effective dose of at least one of aconitine, swatinine, and bergenin. The samples may be mixed at an optimal concentration and then administered to an individual. FIGS. 8-19 below show identification and testing for three above disclosed compounds.

Two additional compounds, orientin and vitexin, were further identified from bioactive fractions of Synatptecx-227 to reduce fibrillization and increase cell survival. The combination of orientin, vitexin, and bergernin was found to have a high cell survival rate of cells that were treated with Aβ.

Referring to FIG. 8 and FIG. 9, FIG. 8 is a preparative high-pressure liquid chromatography (HPLC) image 800 of a first sample (Sample A) of Synaptecx-227 where the Synaptecx-227 has been fractionated into two samples, Sample A and Sample B. Both Sample A and Sample B were further fractionated using preparative high-pressure liquid chromatography (HPLC) into 19 and 23 fractions respectively. All fractions were tested with Aβ₁₋₄₂ fibrils to determine the active fractions. FIG. 9 is an HPLC image 900 of Sample B. Sample A, shown in FIG. 8, was fractionated into fractions A1-A19. Sample B, shown in FIG. 9, was fractionated into fractions B1-B23. Bioactivity of the fractions was determined by a Thioflavin T (THT) assay. Accordingly, focus was made on those bioactive fractions to identify and test the activity of the identified compounds. The identified bioactive compounds were tested in solutions of Aβ to determine an effect of the bioactive compounds. Further testing of an effect of identified bioactive compounds for reducing accumulation of tau protein may further refine and improve the methods and compositions described herein.

As shown by similarly shaded fractions in FIG. 8, fractions A2 and A14 are the active fractions in sample A. The active fractions in FIG. 9 are fractions B1, B2, B3, B4, and B5. Further analysis was performed on the active samples to determine compound(s) that make up the fractions and their efficacity for treating markers of Alzheimer's disease. For instance, solutions of Aβ were treated with the active fractions to determine an effect on Aβ protein and an effective concentration for the fractions. An effective dose may be determined based on the effective concentration of each of the fractions and for combinations of fractions.

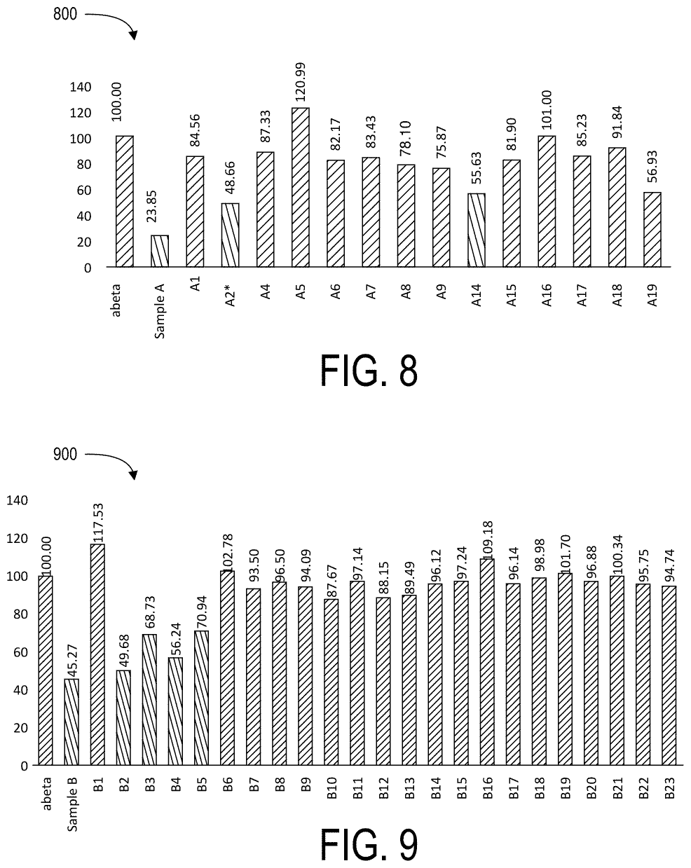

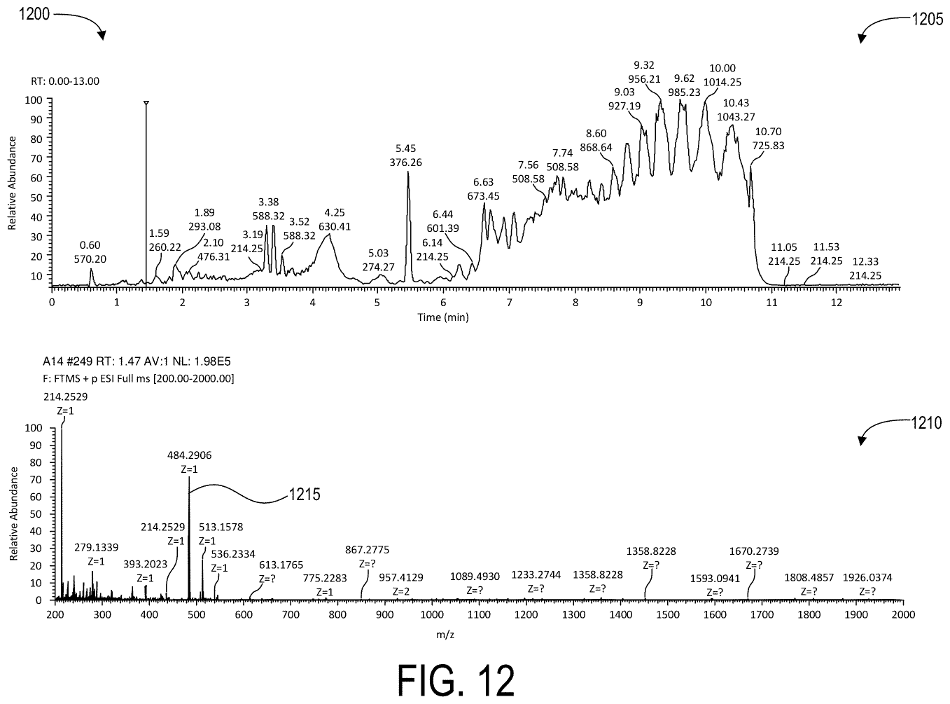

FIGS. 10 and 12 show examples of the LC-MS mass spectrometry (MS) spectra for the A14 fraction taken at various times. The compounds aconitine, swatinine, and bergenin were determined based on further analysis of the various active fractions A2, A14, B1, B2, B3, B4, and B5 via LC-MS and MS. Fragmentation analysis of MS was used to determine probable molecular structure of fractions.

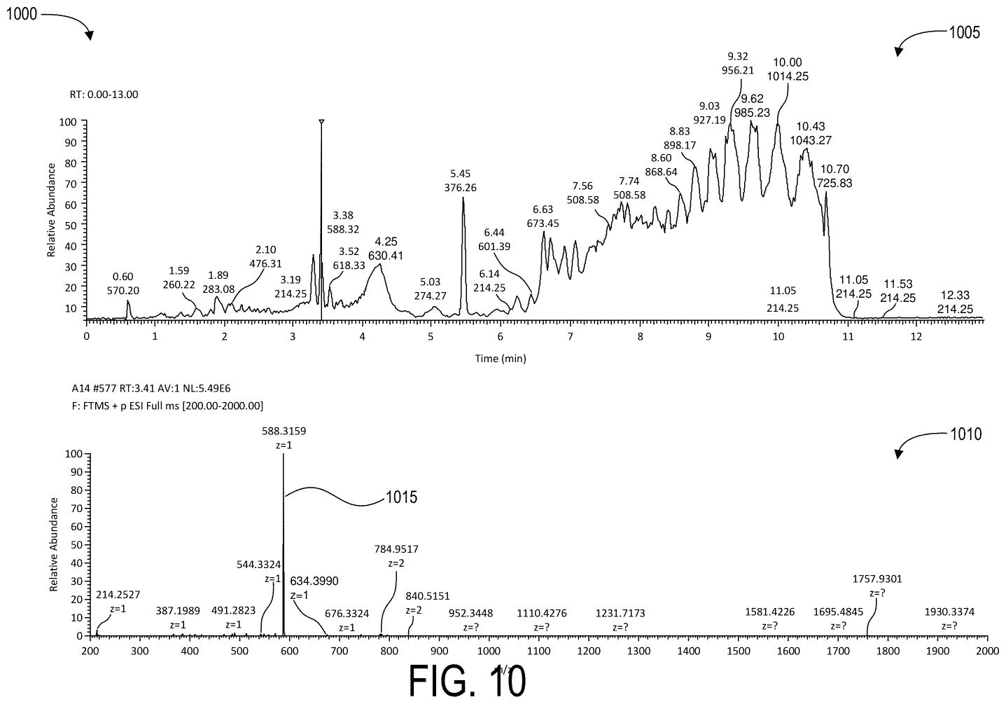

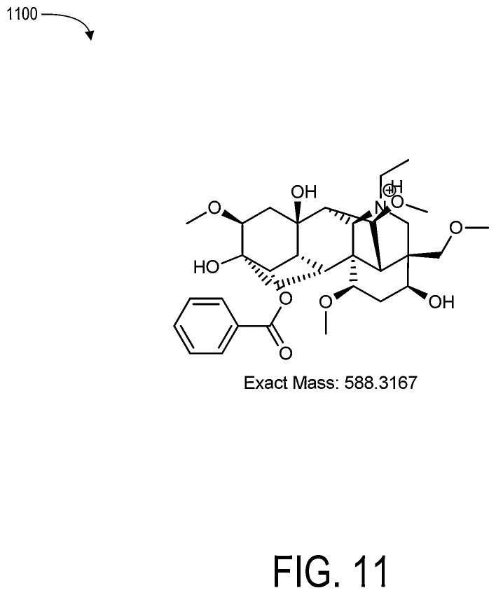

Referring to FIG. 10, FIG. 10 is an LC-MS-mass spectrometry (MS) spectra 1000 for the A14 fraction of Sample A. An LC-MS graph 1005 is shown on top. The bottom of FIG. 10 shows an MS 1010 of a fraction taken at 3.4 minutes on the LC-MS. MS analysis of the 3.4 minute fraction indicates a sharp peak 1015 at a molecular weight of 588. Fragmentation peaks at 544, 491, 387, and 214 are also evident in the MS 1010. Analysis of the fragmentation in the MS 1010 indicates the molecule shown in FIG. 11. FIG. 11 is a probable structure 1100 of the A14 fraction taken at 3.4 minutes on the LC-MS.

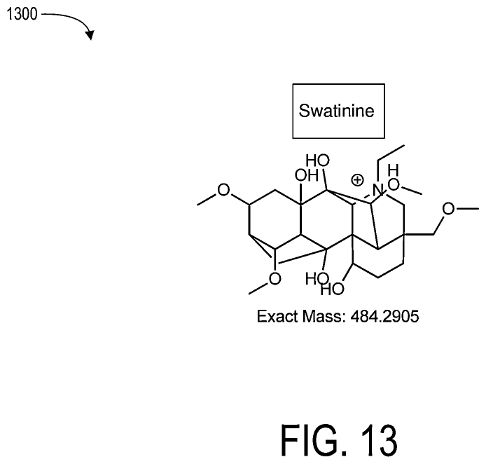

Referring to FIG. 12, FIG. 12 is an LC-MS—mass spectrometry (MS) spectra 1200 for the A14 fraction of Sample A. An LC-MS graph 1205 is shown on top. The bottom of FIG. 12 is an MS 1210 of a fraction taken at 1.5 minutes on the LC-MS. As shown in the MS 1210, analysis of the 1.5 minute fraction indicates a sharp peak 1215 at a molecular weight of 484. FIG. 13 is a probable structure 1300 of the A14 fraction taken at 3.4 minutes on the LC-MS.

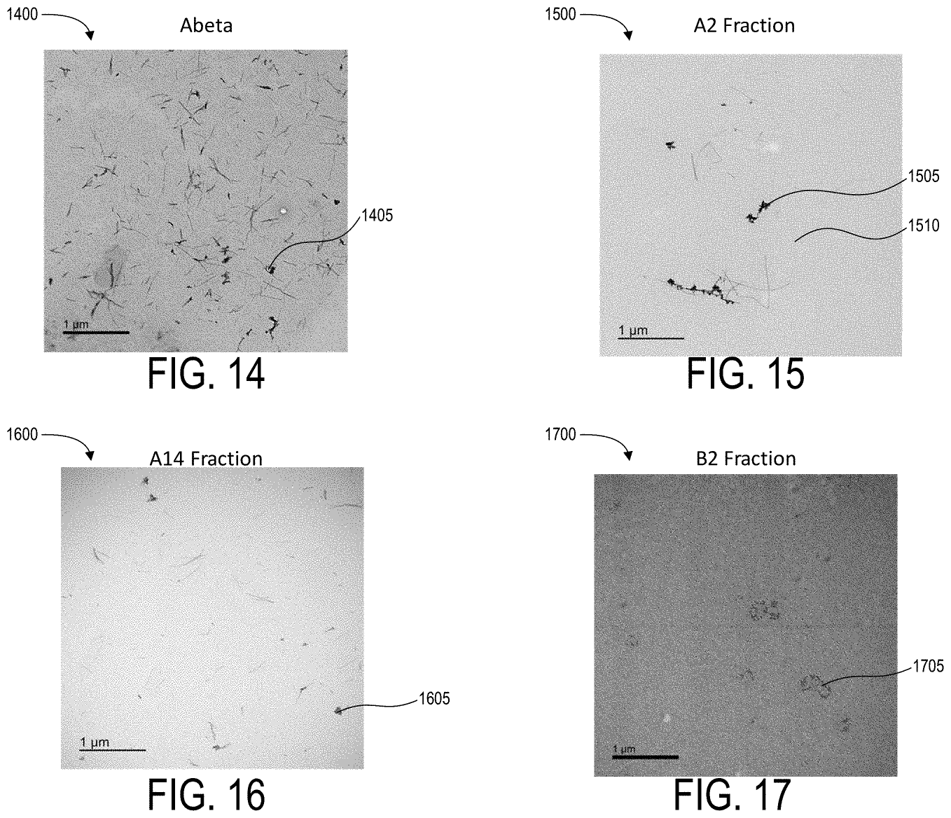

Referring to FIG. 14, FIG. 14 is an electron microscopy image 1400 of Aβ protein. Aggregations 1405 of various size are clearly observable. The scale bar of the image is 1 μm. The image 1400 is useful as a control for trials shown in FIG. 15, FIG. 16, and FIG. 17, whereby the media containing Aβ is also treated with one of the fractions from Sample A or Sample B. FIG. 15 shows an image 1500 of the media containing Aβ and the A2 fraction from Sample A. FIG. 16 shows an image 1600 of the media containing Aβ and the A14 fraction. FIG. 17 shows an image 1700 of the media containing Aβ and the B2 fraction.

The image 1400 shows numerous fibrils of Aβ protein at various sizes. There is a correlation between the presence of Aβ fibrils and Alzheimer's disease. It is believed and reducing the quantity and size of Aβ fibrils may reduce the symptoms and progression of Alzheimer's disease. Aβ protein in its monomeric form is not toxic while Aβ fibrils, which are a hallmark of the Alzheimer's disease, cause neuronal toxicity. The Aβ fibrils may form over time in a solution containing Aβ protein.

Referring to FIG. 15, FIG. 15 is an electron microscopy image 1500 of Aβ protein treated with the A2 fraction from Sample A. The scale bar of the image is 1 μm. When compared with the image 1400 shown in FIG. 14, the image 1500 in FIG. 15 has more space 1510 in between aggregations of the Aβ fibrils 1505. The Aβ fibrils 1505 present in the image 1500 are clearly far fewer in quantity than the Aβ fibrils present in the image 1400 shown in FIG. 14. It can be inferred that one or more compounds in the A2 fraction interfere with Aβ aggregation or otherwise reduce the amount to Aβ fibrils in a sample.

Referring to FIG. 16, FIG. 16 is an electron microscopy image 1600 of Aβ protein and treated with the A14 fraction from Sample A. The scale bar of the image is 1 μm. The image 1600 shows Aβ fibrils 1605 as dark thin structures in the sample. Like the images shown in FIG. 14 and FIG. 15, the image shows the presence of Aβ fibrils 1605 that consists of aggregated Aβ protein. And like the image 1500 shown in FIG. 15, the image 1600 presents fewer Aβ fibrils 1605 than the image 1400 shown in FIG. 14. Accordingly, it can be inferred that one or more compounds in the A14 fraction reduce the incidence of Aβ fibrils in solution.

Referring to FIG. 17, FIG. 17 is an electron microscopy image 1700 of media containing Aβ protein and treated with the B2 fraction from Sample B. The scale bar of the image is 1 μm. The image 1700 shows Aβ fibrils as dark rounded structures 1705 in the sample. As such, the structures 1705 shown in the image 1700 contrast with the thin aggregations shown in FIGS. 14, 15, and 16. Accordingly, it can be inferred from the image 1700 that one or more compounds in the B2 sample interfere with aggregation of the Aβ protein in such a way as to modify the structure that comprises the Aβ fibrils.

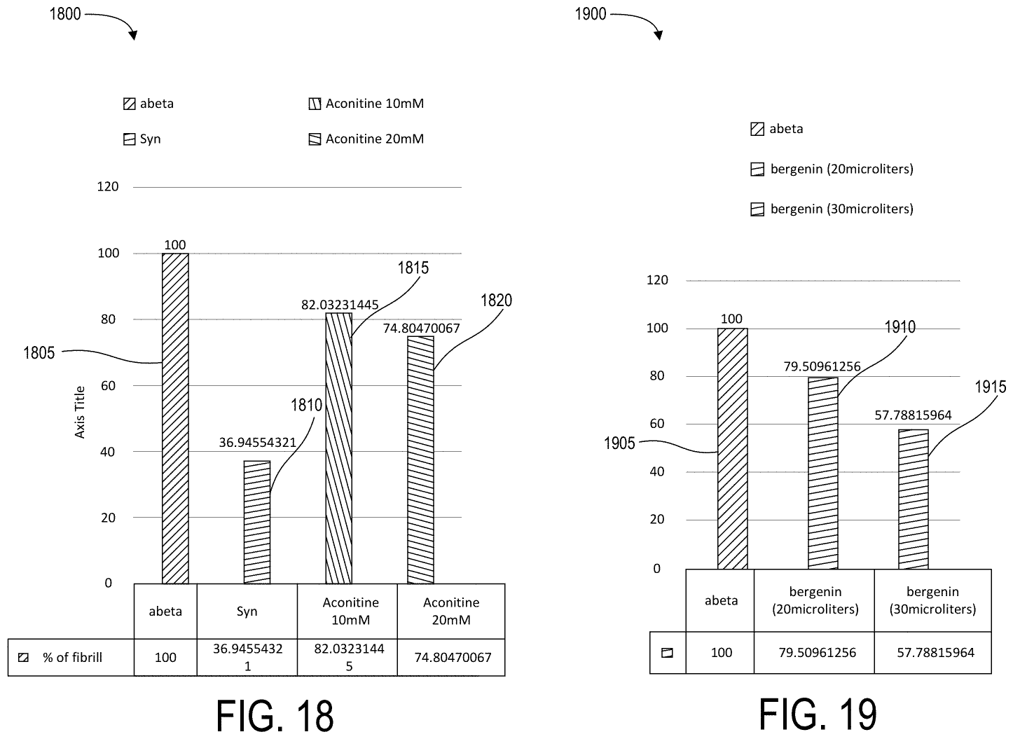

Referring to FIG. 18, FIG. 18 is a bar graph 1800 showing a percentage of Aβ protein fibrillization in various samples. The sample on the left is a control 1805 of Aβ protein. The second sample 1810 from the left is Aβ protein treated with Synaptecx-227. The third sample 1815 from the left is Aβ protein treated with 10 mM aconitine. And the fourth sample 1820 from the left is Aβ protein treated with 20 mM aconitine.

Aconitine was identified as one of the one or more compounds contained in the active fractions labeled A2, A14, B1, B2, B3, B4, and B5 as analyzed by LC-MS/MS from Synaptecx-227. The other thus far identified compounds are swatinine and bergenin. Both aconitine and bergenin were tested against Synaptecx-227 to determine their effectiveness for defibrillation of Aβ fibrils.

The bar graph 1800 may be used to develop an amount and concentration for treating an individual with Alzheimer's disease. The intensity for the control 1805 is normalized to 100. The sample 1810 of Aβ protein treated with Synaptecx-227 shows a substantial decrease in Aβ fibrillization with 36.9% of Aβ fibrillization when compared to the control 1805.

The third sample 1815 includes a concentration of 10 mM aconitine. The percentage of fibrillization for the third sample 1815 is measured at 82.0%, which is lower than the control 1805 and higher than the second sample 1810. The fourth sample 1820 includes a concentration of 20 mM aconitine. The percentage of fibrillization for the fourth sample 1820 is measured at 74.8%, which is lower than the third sample 1815 and significantly higher than the percentage of Aβ fibrillization in the second sample 1810 that contains Synaptecx-227.

Accordingly, it can be inferred from the bar graph 1800 that aconitine reduces the percentage of fibrillization of Aβ protein in media. When the third sample 1815, which has a concentration of 10 mM aconitine, is compared against the fourth sample 1820, which has a concentration of 20 mM aconitine, it can be inferred that increasing the concentration of aconitine to 20 mM results in a lower percentage of Aβ fibrillization. Further, aconitine by itself is not as effective at reducing Aβ fibrillization as Synaptecx-227. Thus, while aconitine has been shown to be one of the components in Synaptecx-227 and has been shown to reduce Aβ fibrillization, there are likely additional compounds in Synaptecx-227 that contribute to the reduction of Aβ fibrillization. As stated above, bergenin was also identified as one of the active compounds in Synaptecx-227. The effectiveness of bergenin for reducing the percentage of Aβ fibrillization in media as compared to a control is shown in FIG. 19.

Referring to FIG. 19, FIG. 19 is a bar graph 1900 showing an amount of Aβ protein in various samples. The sample on the left is a control 1905 of Aβ protein. The second sample 1910 from the left is Aβ protein treated with 200 mM bergenin. The third sample 1915 from the left is Aβ protein treated with 300 mM bergenin.

Like aconitine and swatinine, bergenin was identified as one of the one or more active compounds in Synaptecx-227 that may reduce the formation of Aβ fibrils or otherwise reduce the percentage of Aβ fibrils in a solution. The control 1905 on the left side of the bar graph 1900, which just contains Aβ protein is normalized to 100. The measurement of the percentage of the Aβ protein in the control 1905 that aggregates into Aβ fibrils is compared against solutions containing bergenin.

The second sample 1910 contains 200 mM bergenin. The measurement of Aβ fibrils in solution for the second sample 1910 was 79.3% of the control. Thus, the addition of bergenin resulted in a significant decrease in Aβ fibrillization in solution. The third sample 1915 contained 300 mM bergenin. The measurement of Aβ fibrils in solution for the third sample 1915 was 57.7% of the control.

The measurements taken for the second sample 1910 and the third sample 1915, when compared the control 1905, show that an increase in concentration for bergenin correlates to a decrease in the percentage of Aβ fibrils in solution. Further, the increase in concentration for bergenin, from 200 mM in the second sample 1910 to 300 mM in the third sample 1915 had a nearly linear correlation to the reduction in the percentage of Aβ fibrils in solution. Accordingly, the results in the bar graph 1900 in FIG. 19 show that berginen may be used to reduce an amount of aggregation of Aβ protein, either by preventing the formation of Aβ fibrils or by breaking up aggregated Aβ fibrils.

The concentration for the various compounds including aconitine, bergenin, and swatinine may be combined to build off of one another. For example, aconitine was shown in the bar graph 1800 to reduce the percentage of Aβ fibrils in solution when compared to a control 1805, but was not as effective as Synaptecx-227 at reducing the percentage of Aβ fibrils in solution. An optimal concentration of aconitine may be combined with bergenin, swatinine, and/or other active compounds that are identified and isolated from Synaptecx-227.

For example, aconitine may be combined with bergenin to further reduce the incidence of Aβ fibrillization than either aconitine or bergenin by itself. In an exemplary embodiment, a dose containing a concentration of about 10 mM aconitine and about 200 mM bergenin may be administered to a patient suffering from Alzheimer's disease. In various embodiments, a dose containing a concentration of about 10 mM aconitine and about 300 mM bergenin may be administered to a patient suffering from Alzheimer's disease. In various embodiments, a dose containing a concentration of about 20 mM aconitine and about 200 mM bergenin may be administered to a patient suffering from Alzheimer's disease. In various embodiments, a dose containing a concentration of about 20 mM aconitine and about 300 mM bergenin may be administered to a patient suffering from Alzheimer's disease.

In an exemplary embodiment, swatinine may be administered in an effective dose to treat Alzheimer's disease. In various embodiments, a concentration of swatinine may be combined with about 10 mM aconitine. In various embodiments, a concentration of swatinine may be combined with about 20 mM aconitine. In various embodiments, a concentration of swatinine may be combined with about 200 mM bergenin. In various embodiments, a concentration of swatinine may be combined with about 300 mM bergenin.

In an exemplary embodiment, swatinine may be combined with about 10 mM aconitine and 200 mM bergenin. In various embodiments, swatinine may be combined with about 20 mM aconitine and 200 mM bergenin. In various embodiments, swatinine may be combined with about 10 mM aconitine and 300 mM bergenin. In various embodiments, swatinine may be combined with about 20 mM aconitine and 300 mM bergenin.

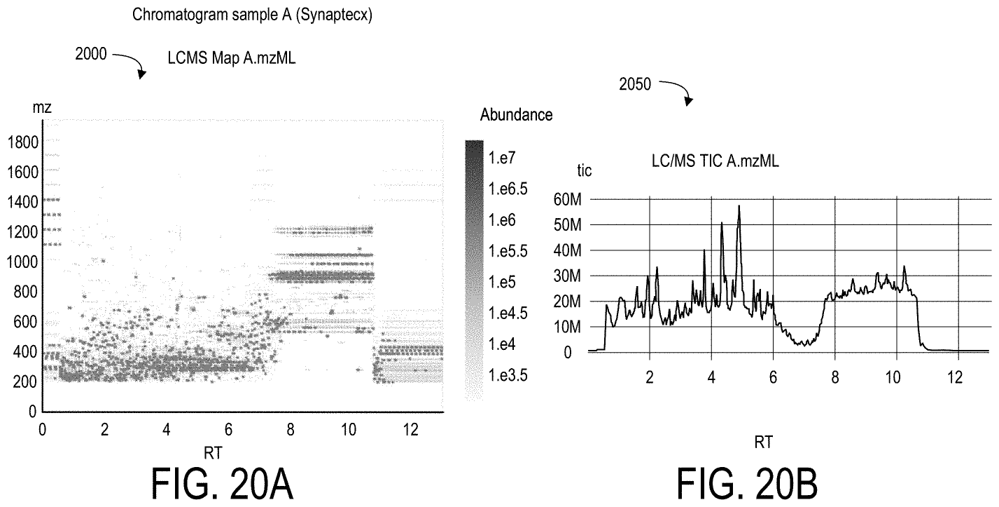

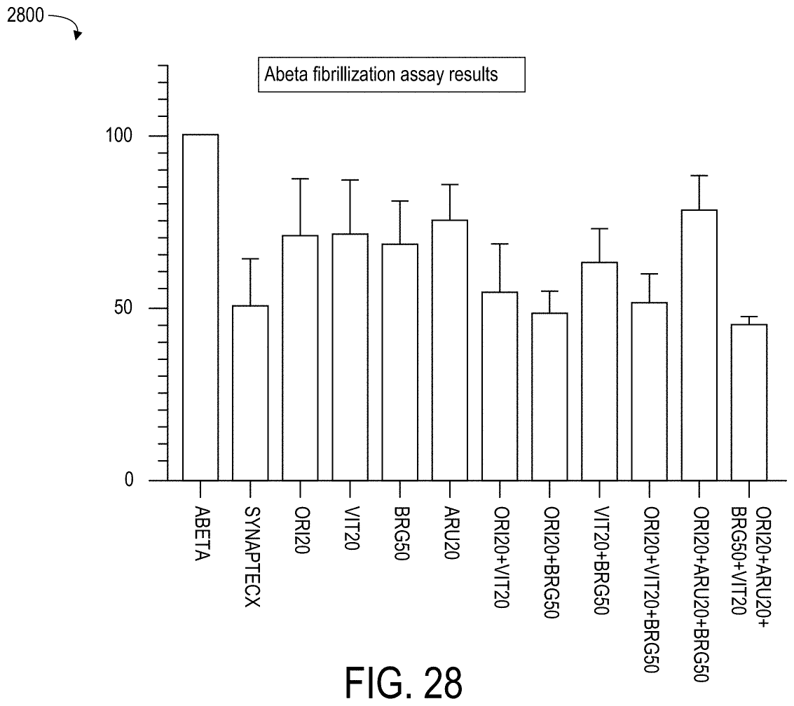

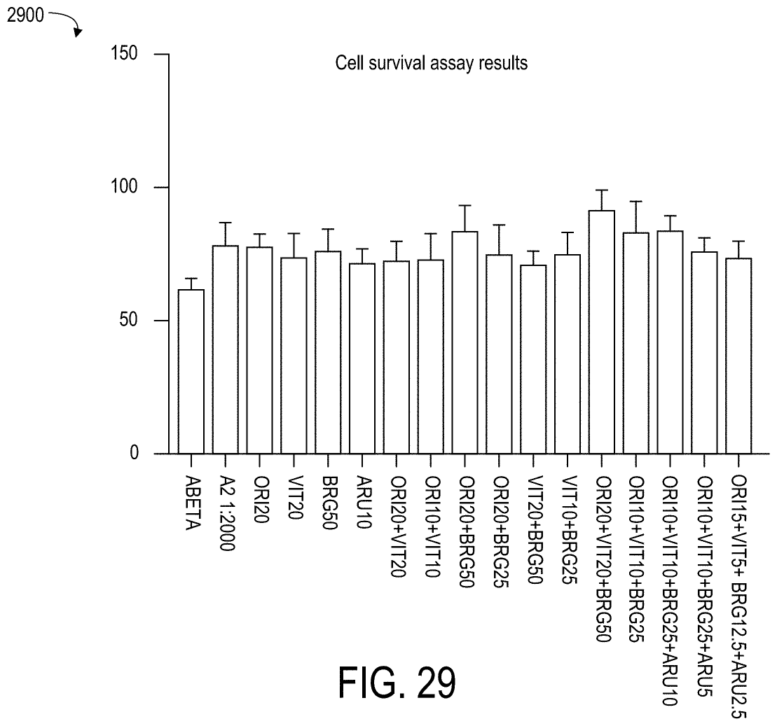

Orientin and vitexin were further identified as bioactive components of Synatptecx-227. FIGS. 20A-27 below show various spectra that were used to isolate and identify orientin and vitexin from Synatptecx-227. FIGS. 28 and 29 show Aβ fibrillization and cell survival assays of various combinations of identified compounds in Synatptecx-227. As such, the combination of orientin, vitexin, and bergenin showed the best results for cell survival and reduced Aβ fibrillization compared to control.

Referring to FIGS. 20A and 20B, FIG. 20A is an LCMS-mass spectrometry (MS) spectra 2000 for a Synaptecx-227 sample (Sample A) showing retention time (rt) in minutes on the x-axis and mass to charge ratio (mz) on the y-axis. FIG. 20B is a chromatogram 2050 of the LCMS shown in FIG. 20A.

Untargeted LC-MS/MS acquisition was performed on a Vanquish Ultrahigh Performance Liquid Chromatography (UPLC) system coupled to a Thermo LTQ Velos Pro (Thermo Fisher Scientific, Bremen, Germany). Chromatographic separation was performed on a Kinetex 1.7 μm 100 Δ pore size C18 reversed phase UHPLC column 50×2.1 mm (Phenomenex, Torrance, Calif.) with a constant flow rate of 0.4 mL/min. The following solvents were used during the LC-MS/MS acquisition: Water with 0.1% Formic Acid (v/v), Optima™ LC/MS Grade, Thermo Scientific™ (solvent A) and acetonitrile with 0.1% Formic Acid (v/v), Optima™ LC/MS Grade, Thermo Scientific™ (solvent B). After injection of 5 uL of sample into the LC system and eluted with linear gradient from 5 to 50% B (0-5 min), 50 to 99% B (5-7 min), 99% B (7-10 min), 99 to 5% B (10-10.1 min), 5% B (10.1-13 min). Data dependent acquisition (DDA) mode was used for acquisition of tandem MS (MS/MS) with default charge state of 1. Full MS was acquired using 1 microscan at a Resolution® of 30 000 at 200 m/z, automatic gain control (ACG) target 5e5, maximum injection time (IT) of 100 ms, scan range 200-2000 m/z and data acquired in profile mode. DDA of MS/MS was acquired using 1 microscan at a Resolution® of 30 000 at 200 m/z, automatic gain control (ACG) target 1e5, top 3 ions selected for MS/MS with isolation window of 1.0 m/z with scan range 200-2000 m/z, fixed first mass of 50 m/z and stepped normalized collision energy (NCE) of 35 eV, minimum ACG target 1e5, dynamic exclusion window of 5 s. Analytical blanks were injected before and after sample injection.

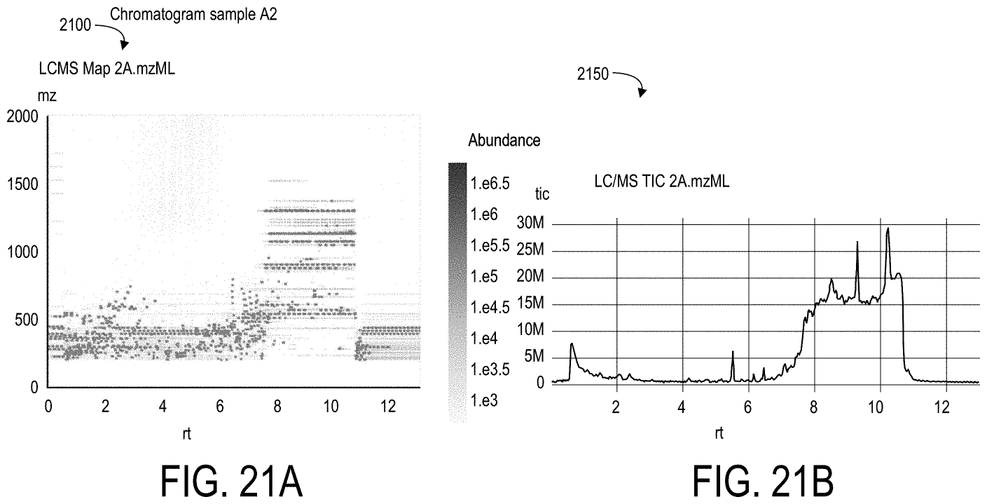

Sample A (Synaptecx-227), shown in FIGS. 20A-20B, was fractionated and collected at 1-minute intervals (A1 to A20). Samples A2 and A3 contained some of the compounds causing bioactivity in Sample A. This was substantiated using bioactivity and cell survival assays. The LCMS spectra for Sample A2 is discussed below in FIGS. 21A-21B.

Referring to FIGS. 21A and 21B, FIG. 21A is an LCMS-mass spectrometry (MS) spectra 2100 for fraction 2 of the Synaptecx-227 sample (Sample A2) showing retention time (rt) in minutes on the x-axis and mass to charge ratio (mz) on the y-axis. FIG. 21B is a chromatogram 2150 of the LCMS shown in FIG. 21A. The chromatogram 2150 in FIG. 21B shows peaks at approximately 0.9 min., 5.5 min., 6.2 min., 6.4 min., and 7.5 min.−10.9 min.

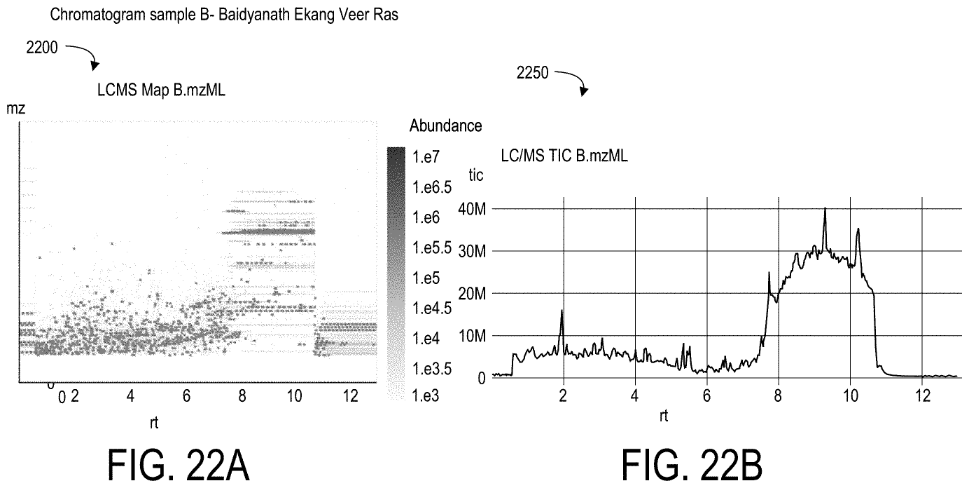

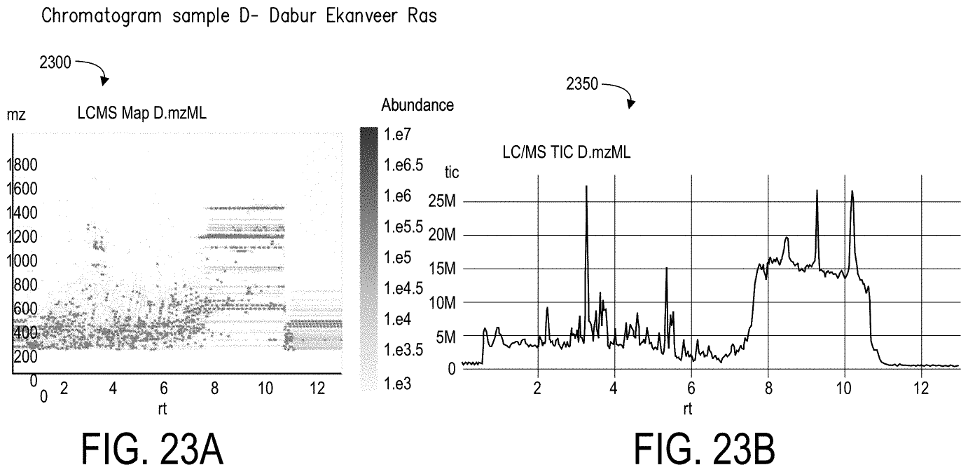

Referring to FIGS. 22A-23B, Ekanveer Ras was identified as possibly containing some of the metabolites causing bioactivity in Sample A. FIGS. 22A-22B show the results of LCMS spectra for a first commercially available sample of Ekanveer Ras. FIGS. 23A-23B show the results of LCMS spectra for a second commercially available sample of Ekanveer Ras.

FIG. 22A is an LCMS-mass spectrometry (MS) spectra 2200 for a first commercially available Ekanveer Ras sample showing retention time (rt) in minutes on the x-axis and mass to charge ratio (mz) on the y-axis. FIG. 22B is a chromatogram 2250 of the LCMS shown in FIG. 22A. The chromatogram in FIG. 21B shows peaks at approximately 2.0 min., 5.5 min., 6.4 min., and 7.5 min.−10.9 min.

FIG. 23A is an LCMS-mass spectrometry (MS) spectra 2300 for a second commercially available Ekanveer Ras sample showing retention time (rt) in minutes on the x-axis and mass to charge ratio (mz) on the y-axis. FIG. 23B is a chromatogram 2350 of the LCMS shown in FIG. 23A. The chromatogram 2350 in FIG. 23B shows fragmentation peaks at approximately 2.3 min., 3.4 min., 5.5 min., 5.7 min., and 7.5 min.−10.9 min.

Using fraction sample A2, sample A3 (not shown), the first commercially available Ekanveer Ras sample, and the second commercially available Ekanveer Ras sample, ingredients were identified using networking analysis. Networking analysis was created using the online workflow at © 2021 Ometa Labs LLC (Release 14.88). Networking analysis relies on spectral similarity of fragmentation spectra (MS2). The precursor ion (MS1) and fragment ions (MS2) mass tolerance were set to 0.02 Da. The network was created by connecting MS2 spectra that have a cosine score above 0.7 and more than 4 matched peaks. A connection between two spectra (visualized as nodes) were kept in the network if and only if each of the nodes appeared in each other's respective top 10 most similar nodes. A maximum size of the molecular family (connected nodes) was set to 100. The spectra in the network were searched against spectral libraries available within the Ometa platform. The library spectra were filtered in the same manner as the input data. All matches kept between network spectra and library spectra were required to have a score above 0.7 and at least 4 matched peaks. The area under the curve for each detected ion was calculated using precursor ion tolerance of 0.02 Da and retention time tolerance of 0.3 min. The mean value was used for quantification purposes. Networking analysis via the Ometa platform can be accessed through the following link: https://demo-flow.ometalabs.online/status?task=0be8c5b3ee4449de8f611e9346de7697.

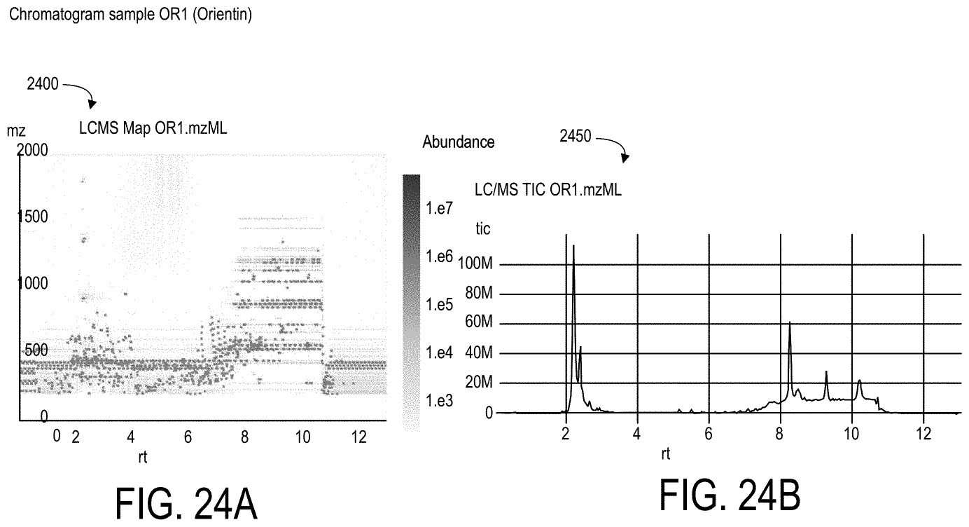

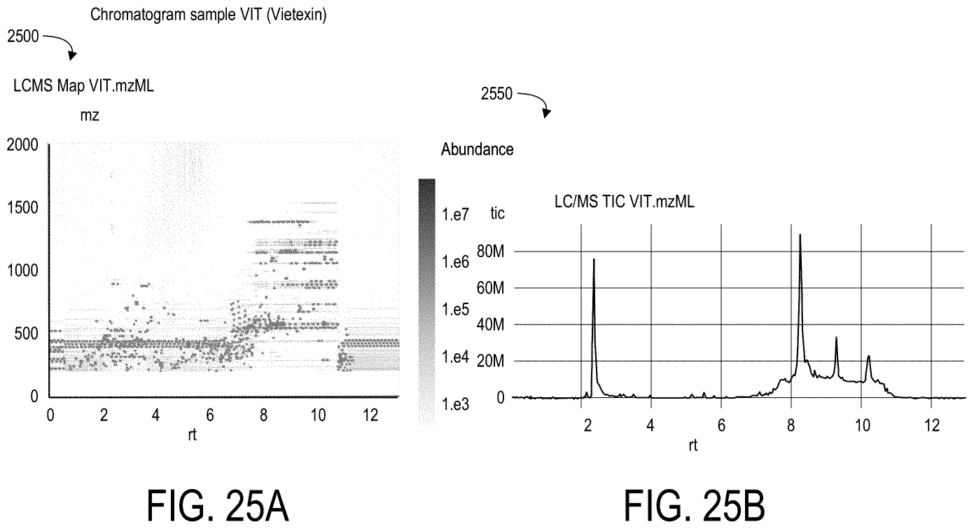

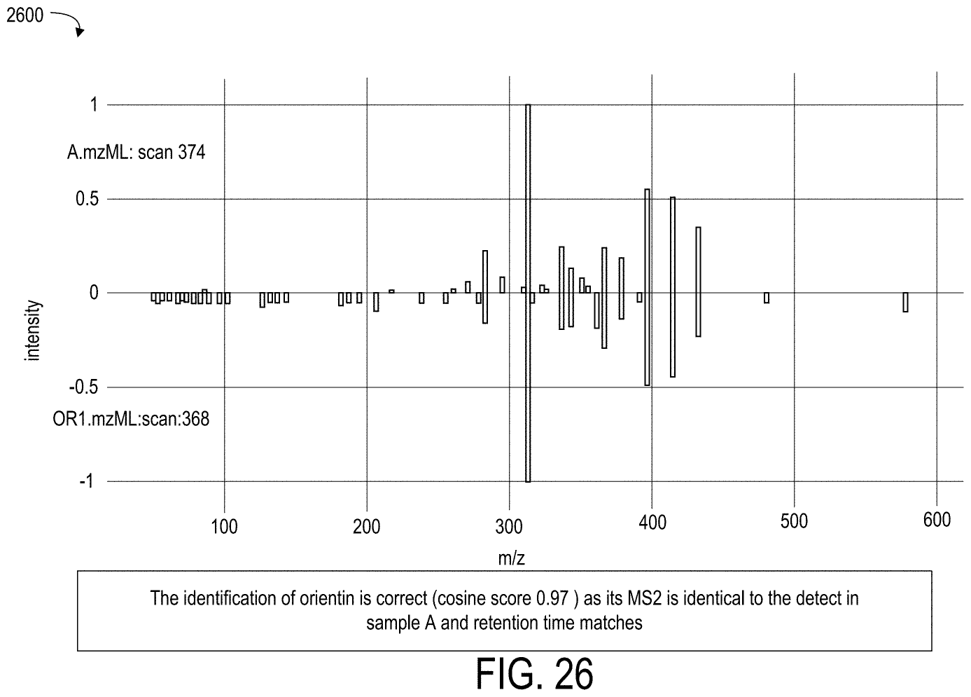

The results of the network analysis showed that orientin, vitexin, and aurantiamide are likely components of Sample A. The peak for orientin is at 2.1 min. The peak for vitexin is at 2.4 min. The peak for aurantiamide is at 5.0 min. FIGS. 24A-27 show LCMS spectra for orientin. FIGS. 25A-25B show LCMS spectra for vitexin. FIGS. 26-7 show side-by-side (mirrored) verification mass spectra comparing the orientin and vitexin portions of sample A to control samples of orientin and vitexin respectively.

Referring to FIGS. 24A and 24B, FIG. 24A is an LCMS-mass spectrometry (MS) spectra 2400 for an orientin sample showing retention time (rt) in minutes on the x-axis and mass to charge ratio (mz) on the y-axis. FIG. 24B is a chromatogram of the LCMS shown in FIG. 24A. The peak for orientin is shown at approximately 2.1 min. Referring to FIGS. 25A and 25B, FIG. 25A is an LCMS-mass spectrometry (MS) spectra 2500 for a vitexin-4-′rhamniside sample showing retention time (rt) in minutes on the x-axis and mass to charge ratio (mz) on the y-axis. The peak for vitexin-4-′rhamniside is at 2.4 min, which is similar to vitexin. FIG. 25B is a chromatogram 2550 of the LCMS shown in FIG. 25A.

Referring to FIG. 26, FIG. 26 shows two mass spectra 2600 for comparison. The first mass spectrograph of the identified orientin portion of Sample A (Synaptecx-227) is displayed from 0 to 1 on the x axis. The second mass spectrograph of commercial orientin is displayed from 0 to −1 on the x-axis.

As shown in FIG. 26, the mass spectra for the orientin portion of Sample A and commercial orientin substantially overlap. The cosine score, which quantifies the similarity between two mass spectra, for the two spectra is 0.97. Accordingly, the composition of the compound at 2.1 min is correctly identified as orientin.

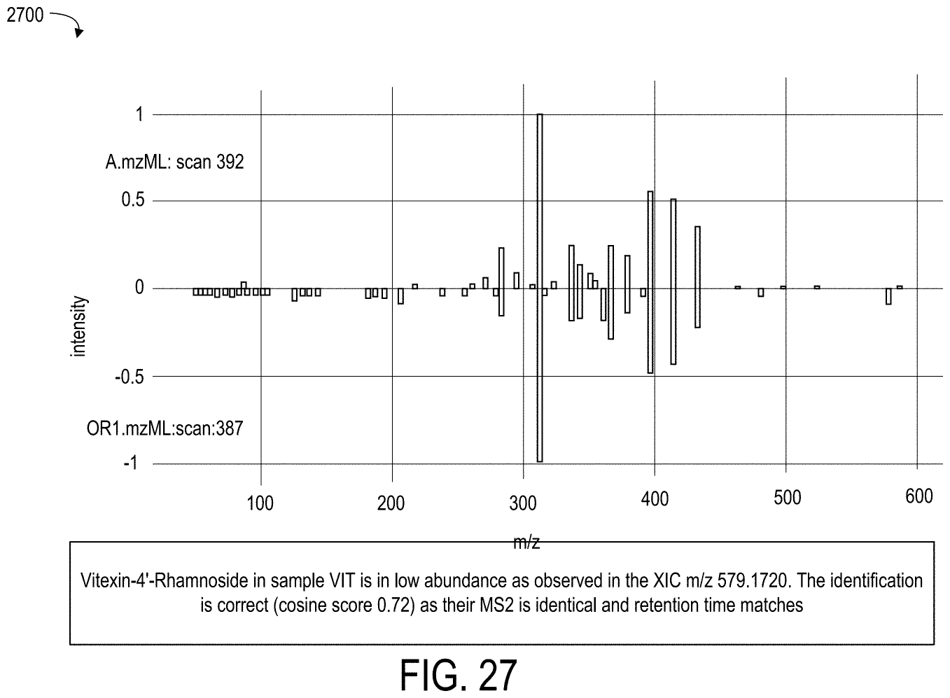

Referring to FIG. 27, FIG. 27 shows two mass spectra 2700 for comparison. The first mass spectra of the identified vitexin portion of Sample A (Synaptecx-227) is displayed from 0 to 1 on the x axis. The second mass spectra of commercial vitexin-4-′rhamnoside is displayed from 0 to −1 on the x-axis.

As shown in FIG. 27, the mass spectra for the vitexin portion of Sample A and commercial vitexin-4-′rhamnoside substantially overlap. The cosine score for the two spectra is 0.72. Accordingly, the composition of the compound at 2.4 min is correctly identified as vitexin.

Referring to FIG. 28, FIG. 28 shows the result of a fibrillization assay 2800 of various compositions of orientin, vitexin, bergenin, and aurantiamide. The control Aβ sample with no treatment shows a 100% fibrillization. The sample treated with a 20 micromolar concentration of orientin decreased Aβ fibrillization by 29.2843% (p value<0.0001 ****). The sample treated with a 20 micromolar concentration of vitexin decreased Aβ fibrillization by 28.8579% (p value<0.0001 ****). The sample treated with a 50 micromolar concentration of bergenin decreased Aβ fibrillization by 31.9532% (p value<0.0001 ****). The sample treated with a 20 micromolar concentration of aurantiamide decreased Aβ fibrillization by 24.8578% (p value<0.0001 ****). The sample treated with a 20 micromolar concentration of orientin and 20 micromolar concentration of vitexin decreased Aβ fibrillization by 45.7211% (p value<0.0001 ****). The sample treated with a 20 micromolar concentration of orientin and 50 micromolar concentration of bergenin decreased Aβ fibrillization by 52.53% (p value<0.0001 ****). The sample treated with a 20 micromolar concentration of vitexin and 50 micromolar concentration of bergenin decreased Aβ fibrillization by 37.29% (p value<0.0001 ****). The sample treated with a 20 micromolar concentration of orientin, 20 micromolar concentration of vitexin, and 50 micromolar concentration of bergenin decreased Aβ fibrillization by 48.9989% (p value<0.0001 ****). The sample treated with a 20 micromolar concentration of orientin, 20 micromolar concentration of aurantiamide and 50 micromolar concentration of bergenin decreased Aβ fibrillization by 21.8175% (p value<0.0001 ****). The sample treated with a 20 micromolar concentration of orientin, 20 micromolar concentration of vitexin, 20 micromolar concentration of aurantiamide and 50 micromolar concentration of bergenin decreased Aβ fibrillization by 55.5057% (p value<0.0001 ****).

Referring to FIG. 29, FIG. 29 shows the result of a cell survival assay 2900 of various compositions of orientin, vitexin, bergenin, and aurantiamide. In the control sample with Aβ treatment, the % of surviving neurons is 61.7592% (p value<0.0001 ****). The next sample from left to right with a 1:2000 dilution of A2/synaptecx had a cell survival of 78.2279%. The next sample with a 20 micromolar concentration of orientin had a cell survival of 77.8195% (p value<0.0001 ****). The next sample of 20 micromolar concentration of vitexin had a cell survival of 73.4208% (p value 0.0019 **). The next sample of 50 micromolar concentration of bergenin had a cell survival of 76.2308% (p value 0.0001 ***). The next sample of 10 micromolar concentration of aurantiamide had a cell survival of 71.5173% (p value 0.0003 ***). The next sample of 20 micromolar concentration of Orientin and 20 micromolar concentration of vitexin had a cell survival of 72.5249% (p value 0.0008 ***). The next sample of 10 micromolar concentration of orientin and 10 micromolar concentration of vitexin had a cell survival of 72.9296% (p value 0.0069 **). The next sample of 20 micromolar concentration of orientin and 50 micromolar concentration of bergenin had a cell survival of 83.5364% (p value<0.0001 ****). The next sample of 10 micromolar concentration of orientin and 25 micromolar concentration of bergenin had a cell survival of 74.8462% (p value 0.0043 **). The next sample of 20 micromolar concentration of vitexin and 50 micromolar concentration of bergenin had a cell survival of 70.5161% (p value 0.0011 **). The next sample of 10 micromolar concentration of vitexin and 25 micromolar concentration of bergenin had a cell survival of 74.6602% (p value 0.0009 ***). The next sample of 20 micromolar concentration of orientin, 20 micromolar concentration of vitexin and 50 micromolar concentration of bergenin had a cell survival of 85.3885% (p value<0.0001 ****). The next sample of 10 micromolar concentration of orientin, 10 micromolar concentration of vitexin, and 25 micromolar concentration of bergenin had a cell survival of 83.2443% (p value<0.0001 ****). The next sample of 10 micromolar concentration of orientin, 10 micromolar concentration of vitexin, 10 micromolar concentration of aurantiamide and 25 micromolar concentration of bergenin had a cell survival of 83.5535% (p value<0.0001 ****). The next sample of 10 micromolar concentration of orientin, 10 micromolar concentration of vitexin, 5 micromolar concentration of aurantiamide and 25 micromolar concentration of bergenin had a cell survival of 75.6103% (p value<0.0001 ****). The next sample of 5 micromolar concentration of orientin, 5 micromolar concentration of vitexin, 2.5 micromolar concentration of aurantiamide and 12.5 micromolar concentration of bergenin had a cell survival of 73.8734% (p value 0.0003 ***).

Accordingly, the highest cell survival was in the sample with a composition of orientin, vitexin, and bergenin. A composition for treatment may include various concentrations of the above listed compounds including orientin and iso-orientin, vitexin, bergenin, and aurantiamide. In an exemplary embodiment, a dose composition containing a concentration of about 10 μM iso-orientin may be administered to a patient suffering from Alzheimer's disease. In various embodiments, a dose composition containing a concentration of about 30 μM iso-orientin may be administered to a patient suffering from Alzheimer's disease. In various embodiments, a dose composition containing a concentration of about 60 μM iso-orientin may be administered to a patient suffering from Alzheimer's disease. In various embodiments, a dose composition containing a concentration of between about 10 μM and 60 μM iso-orientin may be administered to a patient suffering from Alzheimer's disease.

In an exemplary embodiment, a dose composition containing a concentration of about 10 μM orientin may be administered to a patient suffering from Alzheimer's disease. In various embodiments, a dose composition containing a concentration of about 30 μM orientin may be administered to a patient suffering from Alzheimer's disease. In various embodiments, a dose composition containing a concentration of about 60 μM orientin may be administered to a patient suffering from Alzheimer's disease. In various embodiments, a dose composition containing a concentration of between about 10 μM and 60 μM orientin may be administered to a patient suffering from Alzheimer's disease.

In an exemplary embodiment, a dose composition containing a concentration of about 10 μM vitexin may be administered to a patient suffering from Alzheimer's disease. In various embodiments, a dose composition containing a concentration of about 30 μM vitexin may be administered to a patient suffering from Alzheimer's disease. In various embodiments, a dose composition containing a concentration of about 60 μM vitexin may be administered to a patient suffering from Alzheimer's disease. In various embodiments, a dose composition containing a concentration of between about 10 μM and 60 μM vitexin may be administered to a patient suffering from Alzheimer's disease.

In an exemplary embodiment, a dose composition containing a concentration of about 25 μM bergenin may be administered to a patient suffering from Alzheimer's disease. In various embodiments, a dose composition containing a concentration of about 50 μM bergenin may be administered to a patient suffering from Alzheimer's disease. In various embodiments, a dose composition containing a concentration of about 75 μM bergenin may be administered to a patient suffering from Alzheimer's disease. In various embodiments, a dose composition containing a concentration of about 100 μM bergenin may be administered to a patient suffering from Alzheimer's disease. In various embodiments, a dose composition containing a concentration of between about 25 μM and 100 μM bergenin may be administered to a patient suffering from Alzheimer's disease.

In an exemplary embodiment, a dose composition containing a concentration of about 1 μM aurantiamide may be administered to a patient suffering from Alzheimer's disease. In various embodiments, a dose composition containing a concentration of about 5 μM aurantiamide may be administered to a patient suffering from Alzheimer's disease. In various embodiments, a dose composition containing a concentration of about 10 μM aurantiamide may be administered to a patient suffering from Alzheimer's disease. In various embodiments, a dose composition containing a concentration of about 15 μM aurantiamide may be administered to a patient suffering from Alzheimer's disease. In various embodiments, a dose composition containing a concentration of about 20 μM aurantiamide may be administered to a patient suffering from Alzheimer's disease. In various embodiments, a dose composition containing a concentration of between about 1 μM and 20 μM aurantiamide may be administered to a patient suffering from Alzheimer's disease.

In an exemplary embodiment any of the above compositions may be combined. For instance, a dose composition containing a concentration of between about 10 μM and 60 μM iso-orientin and a concentration of between about 10 μM and 60 μM vitexin may be administered to a patient suffering from Alzheimer's disease. In various embodiments, a dose composition containing a concentration of between about 10 μM and 60 μM iso-orientin and a concentration of between about 25 μM and 100 μM bergenin may be administered to a patient suffering from Alzheimer's disease. In various embodiments, a dose composition containing a concentration of between about 10 μM and 60 μM iso-orientin and a concentration of between about 1 μM and 20 μM aurantiamide may be administered to a patient suffering from Alzheimer's disease.

In various embodiments, a dose composition containing a concentration of between about 10 μM and 60 μM orientin and a concentration of between about 10 μM and 60 μM vitexin may be administered to a patient suffering from Alzheimer's disease. In various embodiments, a dose composition containing a concentration of between about 10 μM and 60 μM orientin and a concentration of between about 25 μM and 100 μM bergenin may be administered to a patient suffering from Alzheimer's disease. In various embodiments, a dose composition containing a concentration of between about 10 μM and 60 μM orientin and a concentration of between about 1 μM and 20 μM aurantiamide may be administered to a patient suffering from Alzheimer's disease.

In various embodiments, a dose composition containing a concentration of between about 10 μM and 60 μM vitexin and a concentration of between about 25 μM and 100 μM bergenin may be administered to a patient suffering from Alzheimer's disease. In various embodiments, a dose composition containing a concentration of between about 10 μM and 60 μM vitexin and a concentration of between about 1 μM and 20 μM aurantiamide may be administered to a patient suffering from Alzheimer's disease. In various embodiments, a dose composition containing a concentration of between about 25 μM and 100 μM bergenin and a concentration of between about 1 μM and 20 μM aurantiamide may be administered to a patient suffering from Alzheimer's disease.

In various embodiments, a dose composition containing a concentration of between about 10 μM and 60 μM iso-orientin, a concentration of between about 10 μM and 60 μM vitexin, and a concentration of between about 25 μM and 100 μM bergenin may be administered to a patient suffering from Alzheimer's disease. In various embodiments, a dose composition containing a concentration of between about 10 μM and 60 μM orientin, a concentration of between about 10 μM and 60 μM vitexin, and a concentration of between about 25 μM and 100 μM bergenin may be administered to a patient suffering from Alzheimer's disease. In various embodiments, a dose composition containing a concentration of between about 10 μM and 60 μM iso-orientin, a concentration of between about 10 μM and 60 μM vitexin, and a concentration of between about 1 μM and 20 μM aurantiamide may be administered to a patient suffering from Alzheimer's disease.

In various embodiments, a dose composition containing a concentration of between about 10 μM and 60 μM orientin, a concentration of between about 10 μM and 60 μM vitexin, and a concentration of between about 1 μM and 20 μM aurantiamide may be administered to a patient suffering from Alzheimer's disease. In various embodiments, a dose composition containing a concentration of between about 25 μM and 100 μM bergenin, a concentration of between about 10 μM and 60 μM vitexin, and a concentration of between about 1 μM and 20 μM aurantiamide may be administered to a patient suffering from Alzheimer's disease. In various embodiments, a dose composition containing a concentration of between about 10 μM and 60 μM orientin, a concentration of between about 10 μM and 60 μM vitexin, a concentration of between about 25 μM and 100 μM bergenin, and a concentration of between about 1 μM and 20 μM aurantiamide may be administered to a patient suffering from Alzheimer's disease. In various embodiments, a dose composition containing a concentration of between about 10 μM and 60 μM iso-orientin, a concentration of between about 10 μM and 60 μM vitexin, a concentration of between about 25 μM and 100 μM bergenin, and a concentration of between about 1 μM and 20 μM aurantiamide may be administered to a patient suffering from Alzheimer's disease.

Many variations may be made to the embodiments described herein. For instance, the amounts of the identified active compounds, including but not limited to orientin, vitexin, aurantiamide, aconitine, bergenin, and swatinine, may comprise different concentrations for an effective dose and treatment. All variations are intended to be included within the scope of this disclosure, including combinations of variations. The description of the embodiments herein can be practiced in many ways. Any terminology used herein should not be construed as restricting the features or aspects of the disclosed subject matter. The scope should instead be construed in accordance with the appended claims.

1. A composition, the composition comprising:
an effective dose of at least one of orientin, vitexin and bergenin.

2. The composition of claim 1, wherein the effective dose comprises orientin and vitexin.

3. The composition of claim 1, wherein the effective dose comprises orientin and bergenin.

4. The composition of claim 3, wherein the effective dose further comprises an orientin concentration of between about 10 micromoles per liter to 60 micromoles per liter.

5. The composition of claim 3, wherein the effective dose further comprises a bergenin concentration of between about 25 micromoles per liter to 100 micromoles per liter.

6. The composition of claim 1, wherein the effective dose comprises vitexin and bergenin; and
wherein the effective dose further comprises a vitexin concentration of between about 10 micromoles per liter to 60 micromoles per liter.

7. The composition of claim 1, wherein the effective dose comprises orientin, vitexin, and bergenin.

8. The composition of claim 7, wherein the effective dose further comprises an orientin concentration of between about 10 micromoles per liter to 60 micromoles per liter.

9. The composition of claim 7, wherein the effective dose further comprises a bergenin concentration of between about 25 micromoles per liter to 100 micromoles per liter; and
wherein the effective dose further comprises a vitexin concentration of between about 10 micromoles per liter to 60 micromoles per liter.

10. A method for treatment of a patient, the method comprising:
administering an effective dose of at least one of orientin, vitexin and bergenin.

11. The method of claim 10, wherein the effective dose comprises orientin and vitexin.

12. The method of claim 10, wherein the effective dose comprises orientin and bergenin.

13. The method of claim 12, wherein the effective dose further comprises an orientin concentration of between about 10 micromoles per liter to 60 micromoles per liter.

14. The method of claim 12, wherein the effective dose further comprises a bergenin concentration of between about 25 micromoles per liter to 100 micromoles per liter.

15. The method of claim 10, wherein the effective dose comprises vitexin and bergenin.

16. The method of claim 10, wherein the effective dose comprises orientin, vitexin, and bergenin.

17. The method of claim 16, wherein the effective dose further comprises an orientin concentration of between about 10 micromoles per liter to 60 micromoles per liter.

18. The method of claim 16, wherein the effective dose further comprises a bergenin concentration of between about 25 micromoles per liter to 100 micromoles per liter.

19. A composition for treatment of a patient, the composition comprising: an effective dose comprising orientin, vitexin and bergenin; and
wherein the effective dose further comprises an orientin concentration of between about 10 micromoles per liter to 60 micromoles per liter.

20. The composition of claim 19, wherein the effective dose further comprises a bergenin concentration of between about 25 micromoles per liter to 100 micromoles per liter; and
wherein the effective dose further comprises a vitexin concentration of between about 10 micromoles per liter to 60 micromoles per liter.
